(19) United States
(12) Patent Application Publication
(10) Pub. No.: US 2007/0202005 A1
(43) Pub. Date: Aug. 30, 2007

(54) Method and device for sterilizing medical objects

(75) Inventor: Michael Maschke, Lonnerstadt (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 11/708,149

(22) Filed: Feb. 16, 2007

(30) Foreign Application Priority Data
Feb. 24, 2006 (DE) 10 2006 008 723.2

(51) Int. Cl. A61L 2/24

(52) U.S. Cl. 422/3; 422/62; 340/539.1

(57) ABSTRACT

A method for sterilizing medical objects in a sterilization device is described. A read facility of the sterilization device first reads a machine-readable information code by means of electromagnetic waves from an identification element associated with the medical object. The number of sterilization cycles already undergone by the medical object in question is determined on the basis of the information code. The number of sterilization cycles already undergone is compared with the maximum number of permitted sterilization cycles the medical object in question is allowed to undergo and a warning signal is automatically triggered and/or the function of the sterilization device is restricted, if the number of sterilization cycles undergone reaches or exceeds the maximum number of permitted sterilization cycles. A corresponding sterilization device and a medical object are also described.

### CROSS REFERENCE TO RELATED APPLICATIONS

This application claims priority of German application No. 10 2006 008 723.2 filed Feb. 24, 2006, which is incorporated by reference herein in its entirety.

### FIELD OF THE INVENTION

The invention relates to a method for sterilizing medical objects in a sterilization facility. The invention also relates to a corresponding sterilization device for sterilizing medical objects and a medical object, with which a corresponding sterilization method can be carried out. The medical objects as referred to in this invention can be any medical accessory, medical tools or instruments, such as catheters, endoscopes, needles, drill bits, etc. for example.

### BACKGROUND OF THE INVENTION

The purpose of sterilization is to render an object into a microorganism-free and therefore non-infectious state by killing off possible pathogenic microorganisms. Sterilization is of major importance here, to prevent dangerous and in some circumstances life-threatening infectious diseases in particular in medical practices and hospitals—particularly after intervention during an operation. There are therefore relatively strict rules governing the manner in which certain medical objects should be sterilized. One generally known sterilization method is that of immersing the medical object in question in a sterilizing solution containing corresponding chemicals or washing the object with such a solution. There is also a plurality of further sterilization methods known to the person skilled in the art, such as steam sterilization, hot-air sterilization, gas sterilization, radiation sterilization and plasma sterilization for example. Some of these sterilization methods can have a very major impact on the medical object involved. For example sterilization processes using strong chemicals, UV radiation, radioactive radiation or by means of heat or hot steam or gas exposure frequently result in a greater level of material fatigue, thereby shortening the life of the medical objects. It is therefore stipulated for some medical objects that they can no longer continue to be used after they have undergone a certain number of sterilization cycles, as there is a greater risk that the object in question will be destroyed or fail during subsequent use. This is particularly important in the case of medical objects such as catheters or endoscopes, which are inserted into the human body. In the case of such instruments it is necessary for example to ensure in a reliable manner that part of the object, for example a tip, does not break off, thereby resulting in the risk of injury or parts of objects unintentionally remaining in the body of the patient in an uncontrolled manner.

Until now the staff carrying out the sterilization logged which object had already been sterilized and how often either manually or in a computer-aided manner. To this end the objects are generally identified uniquely by means of labels, engravings, etc., so that a corresponding list can be kept of when and where the respective medical object was sterilized using which sterilization process.

These methods are disadvantageous in that the careful management of corresponding log lists is very time-consuming and there is a risk of incorrect entries, for example if two identical medical objects with similar markings are confused and the entries are therefore not made correctly.

### SUMMARY OF THE INVENTION

One object of the present invention is to provide a method for sterilizing medical objects and a suitable sterilization facility and a corresponding medical object, with which it can be ensured in a less time-consuming yet more reliable manner that a medical object is not sterilized again and further used after exceeding the permitted number of sterilization cycles.

This object is achieved by a method, a sterilization device, and a medical object according to the claims.

With an inventive method for sterilizing medical objects in a sterilization device, a read facility of the sterilization device first reads a machine-readable information code by means of electromagnetic waves from an identification element associated with the medical object. Such an identification element is preferably a transponder, in other words a module with an electronic circuit, which is prompted by means of the electromagnetic waves to transmit the information stored in the circuit. A typical example of such a transponder is what is known as an RFID tag (RFID=Radio Frequency Identification). The transponder does not require a power supply for this. The power is simply obtained from the electromagnetic waves of the read facility.

The information code can hereby allow precise object identification and/or contain information about the type of medical object, for example model, year of manufacture, etc., as well as further details, in particular about the specific sterilization process stipulated for the correct sterilization of the medical object in question. According to the invention this information code is used to determine the number of sterilization cycles, which the medical object in question has undergone. The number of sterilization cycles already undergone is then compared with the maximum number of permitted sterilization cycles that the medical object in question is allowed to undergo. A control facility of the sterilization device then automatically triggers a warning signal and/or restricts the function of the sterilization device, deactivating the sterilization device for example, if the number of sterilization cycles undergone reaches or exceeds the maximum number of permitted sterilization cycles.

It is thus possible in a fully automatic and reliable manner to prevent a medical object, which is not permitted a further sterilization cycle, incorrectly being resterilized and used once again.

A suitable sterilization device for sterilizing medical objects requires a read facility on the one hand, to read a machine-readable information code by means of electromagnetic waves from an identification element associated with a medical object. The sterilization device also requires an evaluation unit, to determine the number of sterilization cycles that the medical object in question has undergone based on the information code, and a comparator unit, to compare the number of sterilization cycles already undergone with the maximum number of permitted sterilization cycles the medical object in question is allowed to undergo. The sterilization device must also be fitted with a corresponding control facility, which is configured such that it automatically triggers a warning signal and/or restricts the function of the sterilization facility, if the number or sterilization cycles undergone reaches or exceeds the maximum permitted number of sterilization cycles. The evaluation unit and comparator unit can thereby also be part of the control facility, as can the read facility.

The dependent claims each contain particularly advantageous embodiments and developments of the invention, it being possible also to develop the dependent claims in particular according to the features of the dependent method claims and vice versa.

There are essentially a number of different possible ways to determine the number of sterilization cycles already undergone and the number of times a certain medical object can be sterilized as a maximum on the basis of the information code.

Thus for example, with one variant of the invention, additional information about the medical object to be sterilized can be determined on the basis of the information code from an external data source, to which the sterilization device is linked, for example a central database of a clinic, a practice, etc., or from the Internet. Information about the medical object can also be sent. It is thus possible for example to store the number of sterilization cycles carried out in a central database and retrieve it from there again. Similarly it is possible to store all the information about the medical object in question in such a database, in particular the maximum number of sterilization cycles permitted for said medical object. Data about the sterilization processes stipulated for the respective medical object during sterilization can also be stored here.

To this end the sterilization device requires a corresponding data interface, for example a link to an intranet bus or to the Internet, in order to be able to retrieve the corresponding information from the required external data source or to send the information.

In one particularly preferred variant however the information code itself contains a first characteristic value representing the number of sterilization cycles the medical object has undergone to date. This characteristic value can for example be a numeral directly, which states the number of sterilization cycles undergone. It can however also for example be a number of flags set within a binary code, etc. When the medical object in question undergoes further sterilization, the characteristic value is automatically overwritten or adjusted by way of a transmit unit of the sterilization device. The characteristic value or number of sterilization cycles can for example be adjusted in instances where the number of sterilization cycles is encrypted in the form of flags set within the information code. A new additional flag is then set correspondingly. Alternatively the information code can be wholly or partially overwritten, to change at least the part of the information code containing the said characteristic value.

With this variant the medical object therefore has an identification element, which contains a machine-readable information code, containing information about the number of sterilization cycles the medical object has already undergone. It is therefore no longer necessary to clarify in an external database—for example on the basis of an identifier—which or how many sterilization cycles the medical object in question has already undergone. This makes the overall process more reliable, as the number of sterilization cycles undergone is associated directly with the medical object.

To achieve maximum reliability, it is also possible to use a combined method. In other words the precise number of sterilization cycles already undergone is stored in each instance directly within the information code in the identification element and can be read from there and updated at any time. This value is then compared with a corresponding value in a central database and a warning signal is output in the event of an incorrect entry.

In order to change the number of sterilization cycles carried out in the information code, the sterilization device requires a corresponding transmit unit, to link a specific information code to a medical object and/or to change an information code linked to a medical object. The identification element must also be configured correspondingly such that the information code can subsequently be changed by means of electromagnetic waves by way of a transmit unit.

A combined read/transmit unit, particularly preferably an RFID read and/or write facility, is used as a preferred alternative to separate read facilities and transmit units for this purpose. The identification element therefore preferably comprises an RFID tag. To produce such an RFID tag only 300 μm to 400 μm thin labels for example are fitted with an extremely flat microchip for the data to be stored—in this instance the information code—and an associated miniature antenna made of copper or aluminum film, which is laminated onto a thin PET carrier film. When a spatially remote read facility transmits an electromagnetic high-frequency field, for example at a frequency of 13.56 MHz, which the RFID tag receives, the stored data is sent back to the read facility on the same path. Similarly the RFID tag can also be written with data. The transmission of information between the RFID tag and the read facility functions without visual contact with a range of one to several meters.

It is quite particularly preferable for the information code also to contain a maximum value, representing the maximum number of permitted sterilization cycles the medical object is allowed to undergo. This maximum value can also be encrypted directly or indirectly in the information code, for example again by means of a simple numeral or a number of flags set in a binary code, etc. This direct storage of the maximum value has the advantage that the sterilization facility or its control facility can immediately verify very simply, by comparing the first characteristic value with the maximum value, whether the maximum value has been reached or exceeded, without requiring access to a database—whether internal or external. As a result the sterilization process can also be carried out according to the invention, if the sterilization device in question has no link to an external central database either temporally or permanently.

The advantage of this method is that the medical object can be sterilized in different sterilization devices—in larger clinics even in different departments—without said sterilization devices having to be networked. Each of the sterilization devices can determine for itself, based on the data contained in the information code, whether a medical object has already undergone the maximum permitted number of sterilization cycles or whether it can be used again.

If however there is corresponding networking of the sterilization device with an external data source, for example an intranet or the Internet, the sterilization facility can also send data to or receive data from an inventory control system or similar program. With a particularly preferred variant of the inventive method a corresponding new medical object is ordered automatically, when the medical object to be sterilized has undergone a specific replacement limit number of sterilization cycles. The replacement limit number of sterilization cycles is thereby of course a function of the maximum number of permitted sterilization cycles. Expediently the replacement limit number is also a function of the time period generally required to repurchase an ordered object of this type and the average frequency of the sterilization cycles, in other words the frequency with which the object is required. In this manner it can be ensured that a new medical object is automatically always available locally in a timely manner, when a specific medical object is no longer allowed to be used because it would exceed the maximum number of permitted sterilization cycles. A warning signal is preferably output to the operator additionally or alternatively, so that said operator is also informed that the object can now no longer be used.

It is obvious that—if an alarm is to be triggered by the control facility—the sterilization device requires a corresponding alarm facility.

The read facility is preferably disposed and configured on the sterilization device such that it automatically captures the information code of a medical object to be sterilized, when the medical object in question is moved into the sterilization area of the sterilization device. For example the read facility can be located in a door area of a sealable disinfection chamber.

It is particularly preferable for the sterilization device to have a sealable disinfection chamber and an activation facility, which automatically activates the read and/or transmit facility, when the disinfection chamber is opened.

The sterilization device advantageously also has a display facility, which is used to display information about a medical object to be sterilized determined on the basis of the information code. This information can be stored directly in the information code or can be retrieved from a database on the basis of the information code. The information displayed can for example include the number of sterilization cycles already undergone and the maximum number of permitted sterilization cycles in addition to details of the type of sterilization process stipulated for the medical object in question (i.e. the operating instructions for sterilizing the medical object in question). Alternatively or additionally the difference between these figures can also be displayed, in other words the number of sterilization cycles the object is still allowed to undergo, so that the operator is always provided with information about the remaining “life” of the medical object in question.

It is particularly advantageous for the control facility to be configured such that it activates the sterilization device automatically as a function of an information code of a medical object to be sterilized captured by the read facility, such that the medical object located in a sterilization area of the sterilization device undergoes a defined sterilization process. For example the control facility can automatically activate the available sterilization units, such as heater, fan, cooler, gas supply, fluid supply, suction, irradiation device etc. appropriately to carry out the required sterilization process in the sterilization area provided, such that the medical object to be sterilized is treated in a specific predetermined manner for a precisely predetermined time, for example being subjected to a specific temperature or a specific irradiation or gas exposure.

In a development of this variant it can also be verified on the basis of the machine-readable information code whether a number of medical objects, which have to undergo different sterilization processes, are located in one sterilization area of the sterilization facility. If such a conflict situation exists, a combined sterilization process (in other words a type of compromise sterilization process) is carried out, which is suitable—where possible—for sterilizing all the medical objects. To find such a combined sterilization process, the control facility can for example select a process sequence from a number of predetermined sterilization processes, with which it is ensured that on the one hand all the medical objects undergo the process steps required for each of them, whilst on the other hand ensuring that one of the medical objects does not require a sub-process, for example a specific irradiation, which could damage another medical object.

Alternatively or additionally a warning signal can also be triggered and/or the function of the sterilization facility is restricted, in other words for example the sterilization facility is taken out of operation completely. When a warning signal is output, the operator can for example remove one of the objects from the sterilization device, thereby resolving the conflict.

It is particularly preferable for the identification element and the read facility also to be used to determine the sub-area of a sterilization area of the sterilization facility in which a specific medical object is located. It is then possible to carry out a sterilization process specified for the medical object in question or a specific sub-process of said sterilization process, for example specific gas exposure or irradiation, in a preferably specific manner in this sub-area. This sub-process is then not carried out in other sub-areas of the sterilization area for example. Corresponding methods, to carry out a location with suitable identification elements, for example RFID tags, already exist. For example it is possible to measure the electromagnetic signals emitted by the identification element by means of a number of receive units within the sterilization facility and to use the signal propagation times and/or signal strengths to determine the position of the medical object in question.

### BRIEF DESCRIPTION OF THE DRAWINGS

The invention is described in more detail below based on exemplary embodiments and with reference to the attached figures, in which:

FIG. 1 shows a schematic diagram of an exemplary embodiment of an inventive sterilization device in the closed state,

FIG. 2 shows a schematic diagram of the sterilization device according to FIG. 1 in the opened state with a medical object located therein,

FIG. 3 shows a detailed schematic diagram of the different functional components of the sterilization device according to FIGS. 1 and 2,

FIG. 4 shows a flow diagram for a possible process sequence during the sterilization of a medical object according to a variant of the present invention, and

FIG. 5 shows a schematic diagram of a sterilization area of a sterilization facility with a number of sub-areas.

### DETAILED DESCRIPTION OF THE INVENTION

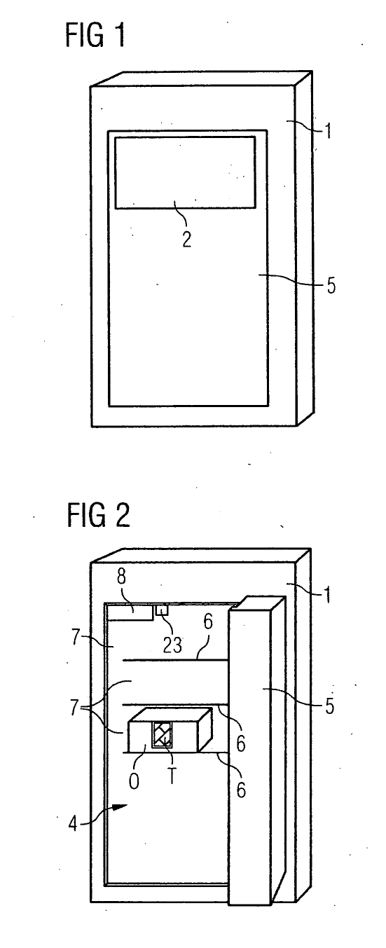

FIG. 1 shows a schematic diagram of a sterilization facility 1 in the form of a sterilization cabinet 1 with a display and control unit 2 disposed outside it, which an operator can use to operate the sterilization facility. A door 5 is located in the front area, which can be used to seal the sterilization chamber 4 within the sterilization cabinet 1.

FIG. 2 shows the sterilization cabinet 1 in the opened state. Inside the sterilization chamber 4 are a number of shelf levels 6 or drawers, which divide the sterilization chamber 4 into a number of sub-areas 7. Inside the sterilization device 1, in this instance in the upper left corner, is an RFID read/write facility 8. Next to this is an activation facility 23, for example a pressure switch, which is used to activate the RFID read/write facility 8 when the door 5 is opened.

Inside the sterilization facility 1 a medical object to be sterilized O is shown schematically in this instance as a box, to which an RFID tag T is attached. An information code C is stored in this RFID tag, containing different information about the medical object O, such as its product name, serial number, manufacturer, date of manufacture, a recommended sterilization program and the number of sterilization cycles already undergone, as well as the maximum number of sterilization cycles the medical object O is allowed to undergo. This information code C is read, as soon as the medical object O is placed in the sterilization chamber 4.

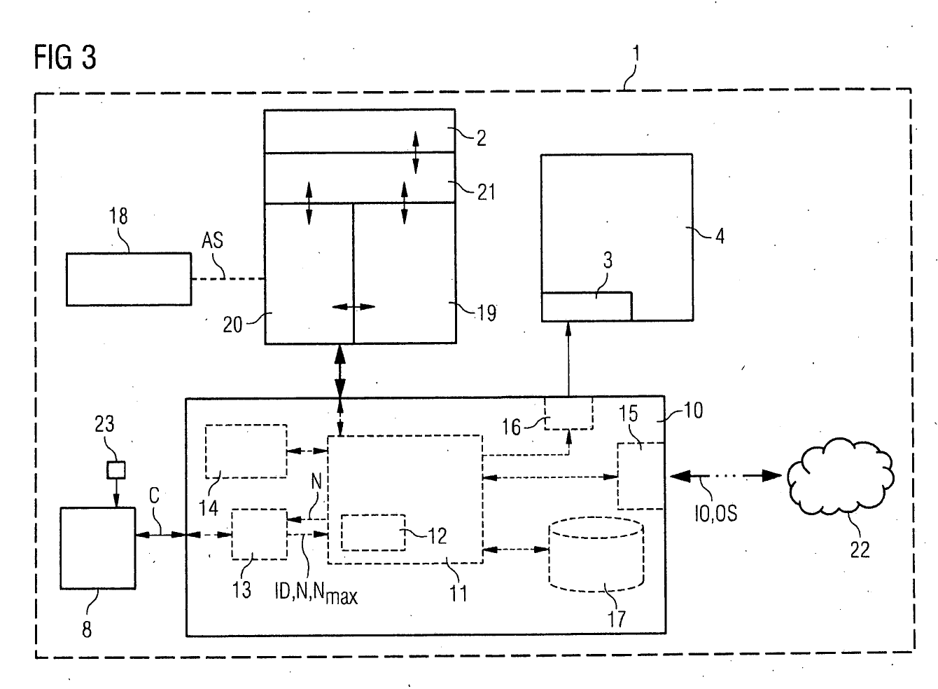

The information code C is then evaluated in a control facility 10 of the sterilization device 1. A corresponding control facility 10 and further functional components of the sterilization device 1 are shown schematically in FIG. 3.

The core element of the control facility 10 in this instance a central unit 11, for example in the form of a microcontroller, on which different sterilization programs, monitoring programs, etc. can be operated. A further component in the control facility 10 is a time measurement facility 14, for example a calendar and/or a clock, which is used to comply with specific process times or even—if this can be found on the basis of the information code—to monitor the maximum service life of the medical object.

The RFID read/write unit 8 is linked to the control facility 10 and is activated automatically by the activation unit 23. The control facility 10 or central unit 11 can prompt the reading of the information code C from an RFID tag T or the writing of an RFID tag T by way of this RFID read/write unit. To this end the control facility 10 has an evaluation and interface unit 13. A read information code C is analyzed or evaluated in this evaluation and interface unit 13 and the information contained therein, such as an identifier ID, the number of sterilization cycles already undergone N and the maximum permitted number Nₘₐₓ of sterilization cycles is transmitted to the central unit 11.

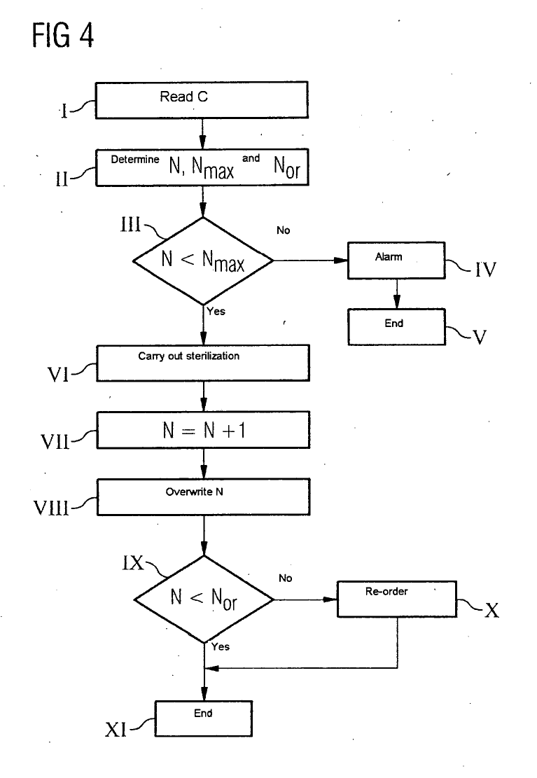

In the exemplary embodiment shown the central unit 11 contains a comparator unit 12 in the form of a program module, which first verifies, before a sterilization process is carried out, whether the number N of sterilization cycles already undergone has exceeded the maximum number Nₘₐₓ of permitted sterilization cycles. The precise sequence of this method is described in more detail below with reference to FIG. 4.

Additional information about the medical object to be sterilized O can be retrieved from a storage unit 17 for already registered objects. The control facility 10 can also communicate by way of an interface 15 with an external data network 22 or an external central database and obtain additional information 10 about the object O or output information in this manner. The sterilization units 3, for example a heating facility, a fan facility, an irradiation facility etc., are activated by the control facility by way of a further interface 16, so that corresponding sterilization processes are carried out within the sterilization chamber 4 or in sub-areas of said sterilization chamber 4. The necessary sterilization units 3 are only shown schematically here in the form of a block.

In order to carry out precisely those sterilization processes, which are suitable for the respective medical objects, it is possible to store sterilization programs intended for specific object types in the storage unit 17 and these are then selected by the central unit 11 based on the information contained in the information code, such as object type, identification, serial number, etc.

The display unit 2 is also linked to the control facility 10 by way of a display interface 20. This display unit 2 is generally controlled by way of a display processor 21. Said display unit 2 is also linked by way of the display processor 21 to a control unit 19, in this instance a touch screen control unit 19, so that inputs can be activated when the operator presses certain display areas. Alternatively or additionally a standard keyboard can also be used and/or voice operation can be enabled with the aid of a voice recognition facility. An acoustic alarm unit 18 is also linked to the display interface 20, so that the control facility can send an alarm signal AS to the acoustic alarm unit 18 by way of the display interface 20, to trigger an alarm, for example if a medical object O placed in the sterilization chamber 4 of the sterilization facility 1 has already exceeded the maximum number Nₘₐₓ of sterilization cycles. A corresponding display simultaneously appears on the display unit 2.

A preferred process sequence for this purpose is shown in FIG. 4. In a first step I the RFID read/write unit first automatically reads the information code C from the RFID tag T on the medical object O, as soon as the medical object O is placed in the sterilization facility 1. In step II the number N of sterilization cycles already undergone, the maximum number Nₘₐₓ of permitted sterilization cycles for the medical object O in question and the replacement limit number Nₒᵣ, the function of which is described further below, are then determined from the information code. In step III it is verified whether the number N of sterilization steps undergone is still below the number Nₘₐₓ of permitted sterilization cycles. If not, no further sterilization can be carried out but in step IV an alarm is triggered and the process is aborted (step V).

Otherwise in step VI the desired sterilization is carried out, with the control facility 10 automatically selecting a suitable sterilization program from a database within the storage unit 17 on the basis of the information contained in the information code C. Alternatively information can also be contained directly within the information code C, about the sterilization process that has to be carried out with the medical object in question O, to ensure correct sterilization.

After sterilization has taken place, the number N of sterilization cycles undergone is increased by one counter in step VII. In step VIII the new value N is written into the RFID tag T on the medical object O. This is done by transmitting the new number N for example from the central unit 11 of the control facility to the evaluation and interface unit 13 and this latter prompting the RFID read/write unit 8 to transmit a corresponding signal to rewrite the RFID tag T on the medical object O (see FIG. 3).

Before the process is terminated in step XI, it is verified again in a process IX, whether the number N of sterilization cycles now undergone is below a replacement limit number Nₒᵣ. If not, in other words if the number N of sterilization cycles undergone has reached said replacement limit number Nₒᵣ, the medical object is automatically reordered in step X. This can be done for example by the central unit 11 automatically sending a corresponding order signal OS by way of the interface 15 to an external network 22 and from there for example to an inventory control system of the clinic or practice where the sterilization device is located. The replacement limit number Nₒᵣ should be selected such that with a normal sequence the new medical object is available locally before the time when the medical object has undergone the maximum number of sterilization cycles, in other words the replacement limit number Nₒᵣ should generally be correspondingly below the maximum number Nₘₐₓ of permitted sterilization cycles. If a new medical object is available very quickly, the replacement limit number Nₒᵣ can however in principle also be equal to the maximum number Nₘₐₓ of permitted sterilization cycles, in other words the new medical object is only ordered, when it has undergone the last permitted sterilization process.

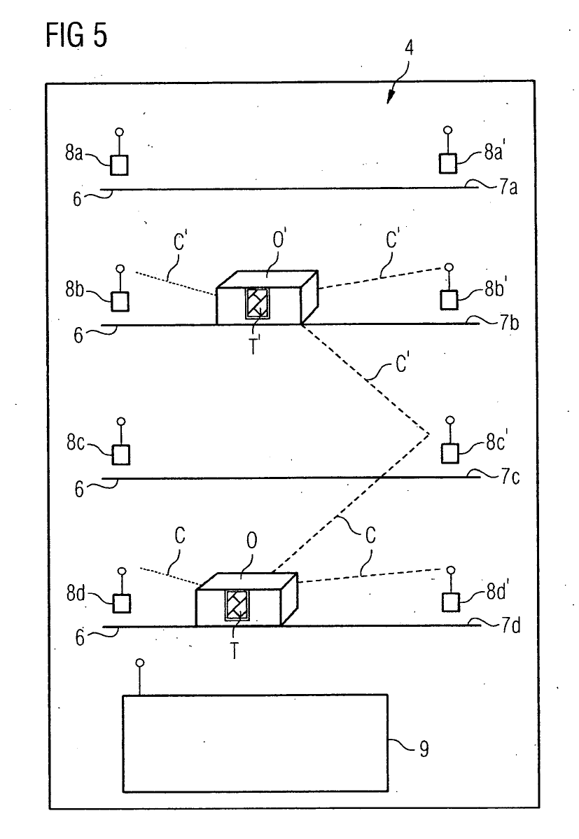

FIG. 5 shows an exemplary embodiment of a particularly advantageous extension of this system. The sterilization area 4 here is divided into different sub-areas 7a, 7b, 7c, 7d at different levels. There are two RFID receiver/transmitters 8a, 8b, 8c, 8d, 8a′, 8b′, 8c′, 8d′ at each of these levels. All the RFID receiver/transmitters 8a, 8b, 8c, 8d, 8a′, 8b′, 8c′, 8d′ are linked to a central RFID control and location unit 9 and together with this form an RFID read and write facility, to read and/or write RFID tags. It is thereby possible to determine, with the aid of the RFID receiver/transmitters 8a, 8b, 8c, 8d, 8a′, 8b′, 8c′, 8d′ positioned at different locations, in which sub-area 7a, 7b, 7c, 7d a specific medical object O′ is located. This can be done for example by evaluating the signal strengths and/or propagation times of the RFID signals received by the RFID receiver/transmitters 8a, 8b, 8c, 8d, 8a′, 8b′, 8c′, 8d′.

It can thus be determined that a first medical object O with a first RFID tag T is located at the lowest level 7d, as the RFID receiver/transmitters 8d, 8d′ and 8c′ receive the strongest signal in respect of said RFID Tag T and corresponding propagation times are present. At the same time it can be determined that a further medical object O′ is located at the second level 7b, as the information code C′ from the RFID tag T′ of this medical object O′ is registered to the strongest degree by the RFID receiver/transmitters 8b, 8b′, 8c′ and the propagation times correspond thereto.

As it can be determined precisely which medical objects are at which level 7a, 7b, 7c, 7d, different sterilization processes can then preferably also be carried out specifically at the different levels 7a, 7b, 7c, 7d. It is thus possible for example to subject the medical objects O, O′ to be sterilized to specific sub-processes, such as specific irradiation or gas exposure, at specific levels, ensuring that the medical objects at the other levels 7a, 7b, 7c, 7d are not affected. It is also possible to display by way of a display unit at which level which medical objects are located. This is particularly expedient in larger sterilization devices. Such geographical identification on the display unit means that in the event of an alarm (e.g. when N=Nₘₐₓ) the object triggering the alarm can be easily located and removed from the sterilization chamber.

To conclude, it should be pointed out once again that the method sequences and sterilization devices described in detail above are exemplary embodiments, which can be modified in many different ways by the person skilled in the art, without departing from the scope of the invention. It is therefore also possible for the control facility to be networked by way of a suitable interface with communication devices other than those mentioned above, for example further PCs, a TV, PDAs, Blackberry devices, mobile telephones, etc. It is thus possible for an operator, who is located at a different station, or even a service technician in the event of a breakdown, to be automatically called to load and unload the sterilization facility.

1.-20. (canceled)

21. A method for sterilizing a medical object in a sterilization device, comprising:
reading a machine readable information code by an electromagnetic wave from an identification element of the medical object via a read unit of the sterilization device;
determining a number of sterilization cycles of the medical object that has undergone to date based on the information code;
comparing the number of sterilization cycles with a maximum number of permitted sterilization cycles that the medical object is allowed to undergo;
automatically triggering a warning signal if the number of sterilization cycles reaches the maximum number of permitted sterilization cycles; and
automatically overwriting the number of sterilization cycles of the medical object if the number of sterilization cycles has not reached the maximum number of permitted sterilization cycles and the medical object undergoes a further sterilization.

22. The method as claimed in claim 21, wherein additional information about the medical object is determined from an external data source based on the information code and is sent to the sterilization device.

23. The method as claimed in claim 21, wherein the information code comprises the maximum number of permitted sterilization cycles that the medical object is allowed to undergo.

24. The method as claimed in claim 21, wherein a corresponding new medical object is automatically ordered when the medical object has undergone a specific replacement limit number of sterilization cycle.

25. The method as claimed in claim 21, wherein the information code of the medical object is automatically captured when the medical object is placed in a sterilization area of the sterilization device.

26. The method as claimed in claim 21, wherein the medical object undergoes a defined sterilization process as a function of the information code.

27. The method as claimed in claim 21,
wherein the sterilization device verifies if a plurality of medical objects are located in one sterilization area of the sterilization device based on information code of each respective medical object,
wherein a combined sterilization process is performed that is suitable to each of the medical objects based on the information code of each respective medical object, and
wherein the combined sterilization process is selected from the group consisting of:
sterilizing, automatically triggering the warning signal, and automatically restricting a function of the sterilization device.

28. The method as claimed in claim 27,
wherein the sterilization area of the sterilization device is divided into a plurality of sub-areas,
wherein a plurality of read units are arranged at the sub-areas, and
wherein locations of the medical objects in the sub-areas are determined based on identification elements of the medical objects and the read units arranged at the sub-areas.

29. The method as claimed in claim 21, wherein information about the medical object determined based on the information code is displayed on a display unit.

30. The method as claimed in claim 21, wherein the warning signal is automatically triggered if the number of sterilization cycles exceeds the maximum number of permitted sterilization cycles.

31. The method as claimed in claim 21, wherein a function of the sterilization device is restricted if the number of sterilization cycles reaches or exceeds the maximum number of permitted sterilization cycles.

32. A sterilization device for sterilizing a medical object, comprising:
a read unit that reads a machine readable information code by an electromagnetic wave from an identification element associated with the medical object;
an evaluation unit that determines a number of sterilization cycles that the medical object has undergone based on the information code;
a comparator unit that compares the number of sterilization cycles with a maximum number of permitted sterilization cycles that the medical object is allowed to undergo;
a control unit that automatically triggers a warning signal if the number of sterilization cycles exceeds the maximum number of permitted sterilization cycles; and
a transmit unit that changes the information code of the medical object if the number of sterilization cycles has not exceeded the maximum number of permitted sterilization cycles and the medical object undergoes a further sterilization.

33. The sterilization device as claimed in claim 32, further comprising:
a data interface that determines additional information about the medical object from an external data source based on the information code and the additional information is sent to the sterilization device,
a display unit that displays the information about the medical object, and
a sealable disinfection chamber and an activation unit that automatically activates the read unit when the disinfection chamber is opened.

34. The sterilization device as claimed in claim 32, wherein the read unit is an RFID unit and automatically captures the information code of the medical object when the medical object is placed in a sterilization area of the sterilization device.

35. The sterilization device as claimed in claim 32, wherein the control unit activates the sterilization device as a function of the information code of the medical object such that the medical object located in a sterilization area of the sterilization device undergoes a defined sterilization process.

36. The sterilization device as claimed in claim 32, wherein the warning signal is automatically triggered if the number of sterilization cycles exceeds the maximum number of permitted sterilization cycles.

37. The sterilization device as claimed in claim 32, wherein a function of the sterilization device is restricted if the number of sterilization cycles reaches or exceeds the maximum number of permitted sterilization cycles.

38. A medical object to be sterilized, comprising:
an identification element that comprises a machine readable information code,
wherein the information code comprises a number of sterilization cycles that the medical object has undergone, and
wherein the information code is subsequently changed by an electromagnetic wave.

39. The medical object as claimed in claim 38, wherein the information code comprises a maximum number of permitted sterilization cycles the medical object is allowed to undergo and comprises information to control a sterilization process to sterilize the medical object.

40. The medical object as claimed in claim 38, wherein the identification element comprises an RFID tag.
